(19) United States
(12) Patent Application Publication
(10) Pub. No.: US 2022/0262516 A1
(43) Pub. Date: Aug. 18, 2022

(54) Atrial Fibrillation Prediction Model And Prediction System Thereof

(71) Applicant: China Medical University Hospital, Taichung City (TW)

(72) Inventors: Tzung-Chi Huang, Taichung City (TW); Ken Ying-Kai Liao, Taichung City (TW); Kuan-Cheng Chang, Taichung City (TW)

(73) Assignee: China Medical University Hospital, Taichung City (TW)

(21) Appl. No.: 17/628,991

(22) PCT Filed: Sep. 6, 2019
(86) PCT No.: PCT/CN2019/104724

§ 371 Date: Jan. 21, 2022

(51) Int. Cl. G16H 50/20; A61B 5/318; A61B 5/308

(52) U.S. Cl. CPC G16H 50/20; A61B 5/308; A61B 5/318

(57) ABSTRACT

An atrial fibrillation prediction system is provided. The atrial fibrillation prediction system includes an electrocardiogram obtaining unit and a non-transitory machine-readable medium. The non-transitory machine-readable medium is configured for storing a program which is executed by a processing unit to obtain a prediction result. The program includes a reference database obtaining module, a reference feature selecting module, a training module, a target feature selecting module and a comparing module.

### RELATED APPLICATIONS

This application is a National Stage Entry of International Application No. PCT/CN2019/104724, filed Sep. 6, 2019, the content of which is incorporated herein by reference.

### TECHNICAL FIELD

The present disclosure relates to a medical information analysis model and system. More particularly, the present disclosure relates to an atrial fibrillation prediction model and an atrial fibrillation prediction system.

### DESCRIPTION OF RELATED ART

Atrial fibrillation is a disease in which the heart beats irregularly and often too fast because the function of generating rhythm signals in the heart is abnormal, and in which the heartbeat can reach 350 beats per minute. Atrial fibrillation is the most common abnormal heart rhythm. On average, 1 out of every 100 people in the entire population suffers from atrial fibrillation, and the proportion of suffering from atrial fibrillation increases as the age increases. Among the people over 60, 4 out of every 100 people suffer from atrial fibrillation. Among the people over 80, 1 out of every 10 people suffers from atrial fibrillation. In 2010, it is estimated that 33.5 million people suffered from atrial fibrillation worldwide. In addition, there may be many patients who have not been diagnosed because they have no symptoms. It is estimated that the number of patients with atrial fibrillation in Asia will reach 72 million by 2050.

Patients with atrial fibrillation have the risk 5 times higher than ordinary people to develop thrombotic infarction diseases, including stroke, pulmonary embolism and peripheral vascular embolism. In the past studies, it has shown that among the patients suffering from atrial fibrillation, patients with paroxysmal atrial fibrillation have lower stroke rate than patients with persistent atrial fibrillation. The prognosis of patients with persistent atrial fibrillation after a stroke is worse than patients with paroxysmal atrial fibrillation, and patients with persistent atrial fibrillation also have a higher risk of subsequent stroke. Therefore, the pattern of atrial fibrillation and stroke are highly correlated. It is estimated that the number of patients suffering from stroke caused by atrial fibrillation in Asia will reach 2.9 million by 2050. Patients with persistent atrial fibrillation have higher stroke rate than patients with paroxysmal atrial fibrillation, and the prognosis thereof after a stroke is also worse. Clinically, CHA2DS2-VASc score is mainly used to assess the risk of stroke in patients with atrial fibrillation. The parameters evaluated in CHA2DS2|VASc score include age, gender and comorbidities, which includes infarction disease, hypertension, congestive heart failure, diabetes and vascular disease. As CHA2DS2-VASc score increases, the risk of vascular embolism also gradually increases. However, there has been no research on the correlation between the electrocardiogram characteristics of patients with atrial fibrillation and stroke so far.

The electrocardiogram provides information of atrial fibrillation, such as the frequency and pattern thereof during the day. However, the data is huge and cannot be manually analyzed. Therefore, in the conventional technology, it is unable to efficiently analyze the large amount of data, so as for helping physicians to further judge whether a subject is a patient with atrial fibrillation and stroke in clinical practice and to improve the accuracy of detection.

### SUMMARY

According to an aspect of the present disclosure, an atrial fibrillation prediction model includes establishing steps as follows. A reference database is obtained, a feature selecting step is performed and a training step is performed. The reference database includes a plurality of reference twelve-lead electrocardiogram signal sequences. In the feature selecting step, at least one feature value is selected according to the reference database. The at least one feature value includes an image interval where an electrocardiogram signal curvature changes the most obtained by calculating a peak-to-peak time difference in the reference twelve-lead electrocardiogram signal sequences with a calculating unit. In the training step, an electrocardiogram signal real-time value is stored by a long short term memory (LSTM) and a correlation between the at least one feature value and the electrocardiogram signal real-time value is calculated. The long short term memory is updated when the correlation exceeds a first preset threshold, and the atrial fibrillation prediction model is obtained when training reaches convergence and a preset result is obtained.

According to another aspect of the present disclosure, an atrial fibrillation prediction system includes an electrocardiogram obtaining unit and a non-transitory machine-readable medium. The electrocardiogram obtaining unit is configured for obtaining a target twelve-lead electrocardiogram signal sequence. The non-transitory machine-readable medium is connected to the electrocardiogram obtaining unit by at least one signal, and the non-transitory machine-readable medium is configured for storing a program. The program is executed by at least one processing unit to obtain a prediction result, and the program includes a reference database obtaining module, a reference feature selecting module, a training module, a target feature selecting module and a comparing module. The reference database obtaining module is configured for obtaining a reference database, and the reference database includes a plurality of reference twelve-lead electrocardiogram signal sequences. The reference feature selecting module is configured for selecting at least one reference feature value according to the reference database. The at least one reference feature value includes an image interval where an electrocardiogram signal curvature changes the most obtained by calculating a peak-to-peak time difference in the plurality of reference twelve-lead electrocardiogram signal sequences with a calculating unit. The training module includes a long short term memory (LSTM). The long short term memory is configured for storing an electrocardiogram signal real-time value and calculating a correlation between the at least one reference feature value and the electrocardiogram signal real-time value. The long short term memory is updated when the correlation exceeds a first preset threshold, and an atrial fibrillation prediction model is obtained when training reaches convergence. The target feature selecting module is configured for analyzing the target twelve-lead electrocardiogram signal sequence to obtain a target feature value. The target feature value includes an image interval where a target electrocardiogram signal curvature changes the most obtained by calculating a peak-to-peak time difference in the target twelve-lead electrocardiogram signal sequence with another calculating unit. The comparing module is configured for analyzing and comparing the target feature value and the at least one reference feature value with the atrial fibrillation prediction model, so as to obtain a preset result.

### BRIEF DESCRIPTION OF THE DRAWINGS

The present disclosure can be more fully understood by reading the following detailed description of the embodiment, with reference made to the accompanying drawings as follows:

FIG. 1 is a flow chart of establishing steps of an atrial fibrillation prediction model according to an embodiment of the present disclosure.

FIG. 2 is a block diagram of an atrial fibrillation prediction system according to another embodiment of the present disclosure.

FIG. 3 is a schematic diagram of data labeling platform of a reference database of the atrial fibrillation prediction model of the present disclosure.

FIG. 4 is a schematic diagram of a structure of a long short term memory of the atrial fibrillation prediction model of the present disclosure.

FIG. 5 is a structural diagram of the long short term memory of the atrial fibrillation prediction model of the present disclosure.

FIG. 6 is a diagram of receiver operating characteristic curve for predicting a subject's stroke rate of the atrial fibrillation prediction system of the present disclosure.

### DETAILED DESCRIPTION

The present disclosure will be further exemplified by the following specific embodiments. However, the readers should understand that the present disclosure should not be limited to these practical details thereof, that is, in some embodiments, and these practical details are used to describe how to implement the materials and methods of the present disclosure and are not necessary.

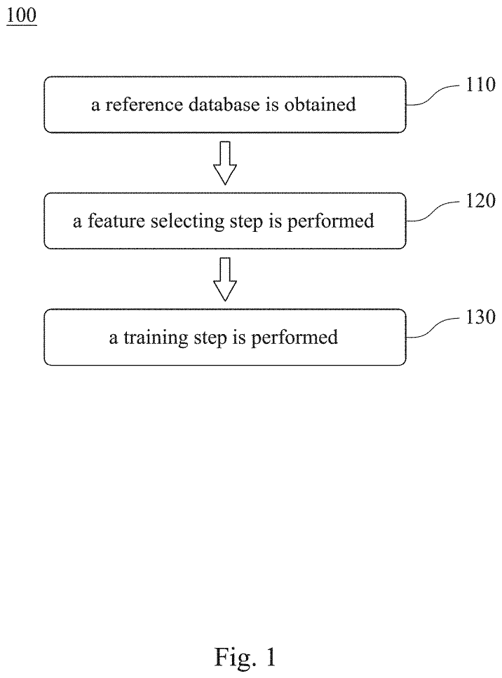

Please refer to FIG. 1, which is a flow chart of establishing steps of an atrial fibrillation prediction model 100 according to an embodiment of the present disclosure. The establishing steps of the atrial fibrillation prediction model 100 of the present disclosure include Step 110, Step 120 and Step 130. The established atrial fibrillation prediction model can be used to predict a stroke rate of a subject.

In Step 110, a reference database is obtained. The reference database includes a plurality of reference twelve-lead electrocardiogram signal sequences. Furthermore, the reference twelve-lead electrocardiogram signal sequences can be preliminary classified into an abnormal data and a non-abnormal data and labeled, so as to divide the reference database into two categories.

In Step 120, a feature selecting step is performed to select at least one feature value according to the reference database. The feature value includes an image interval where an electrocardiogram signal curvature changes the most obtained by calculating a peak-to-peak time difference in the reference twelve-lead electrocardiogram signal sequences with a calculating unit.

In Step 130, a training step is performed to store an electrocardiogram signal real-time value by a long short term memory (LSTM) and calculate a correlation between the feature value and the electrocardiogram signal real-time value. The long short term memory is updated when the correlation exceeds a first preset threshold, and the atrial fibrillation prediction model is obtained when training reaches convergence and a preset result is obtained. The long short term memory can further include a forget gate, an input gate and an output gate. The forget gate is to filter the electrocardiogram signal real-time value whose curvature changes excessively to obtain an input value. The input gate is to input the input value, and the correlation is calculated by a Sigmoid function. The output gate is to calculate the correlation by the Sigmoid function to obtain an output value, and the output value is added to the long short term memory when the output value exceeds a second preset threshold. Preferably, the forget gate, the input gate and the output gate can be concatenated bi-directionally, and the first preset threshold and the second preset threshold can be determined by a tan h function. The long short term memory can be a bi-directional long short term memory (bi-directional LSTM).

The first preset threshold and the second preset threshold are determined by the tan h function. The output value of the tan h function is between −1 and 1, which is the preset value from calculating a large number of twelve-lead electrocardiogram signal sequences with mathematical formula of machine learning. During the training of the atrial fibrillation prediction model, when the correlation between the feature value and the electrocardiogram signal real-time value exceeds the first preset threshold, the long short term memory is updated to reach convergence and obtain the atrial fibrillation prediction model. When the correlation is closer to −1, the probability that the subject does not have atrial fibrillation is higher. On the contrary, when the correlation is close to 1, the probability that the subject has atrial fibrillation is higher. When predicting whether the subject has atrial fibrillation with the atrial fibrillation prediction model, the forget gate will first filter the electrocardiogram signal real-time value to obtain an input value, and the correlation calculated by the Sigmoid function is input through the input gate. The output gate will calculate the correlation by the Sigmoid function to obtain the output value. When the output value exceeds the second preset threshold, the output value is added to the long short term memory. When the output value is closer to −1, the probability that the subject does not have atrial fibrillation is higher. On the contrary, when the output value is close to 1, the probability that the subject has atrial fibrillation is higher.

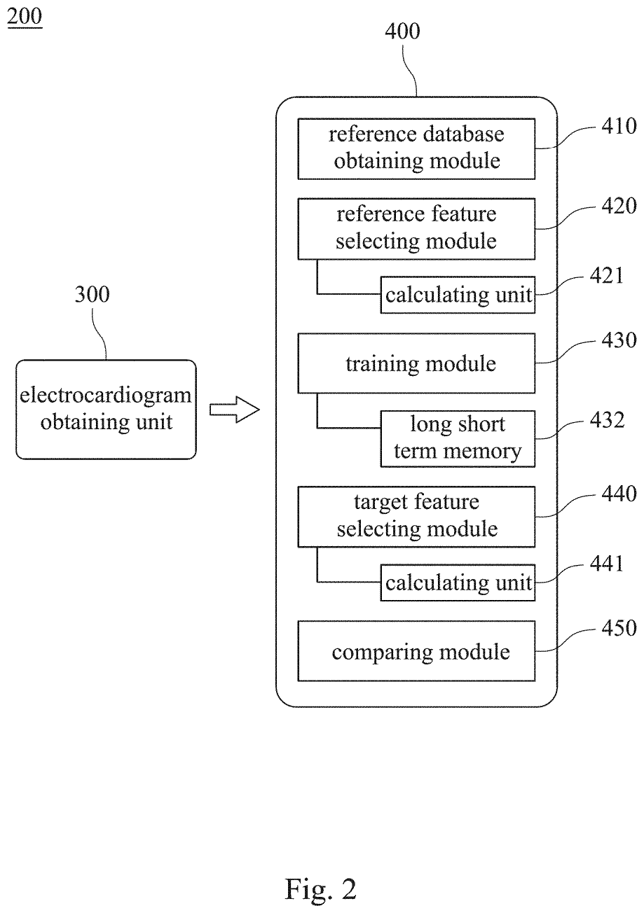

Please refer to FIG. 2, which is a block diagram of an atrial fibrillation prediction system 200 according to another embodiment of the present disclosure. The atrial fibrillation prediction system 200 of the present disclosure includes an electrocardiogram obtaining unit 300 and a non-transitory machine-readable medium 400. The atrial fibrillation prediction system 200 can be used to predict a stroke rate of a subject.

The electrocardiogram obtaining unit 300 is configured for obtaining the target twelve-lead electrocardiogram signal sequence of the subject, and obtaining the reference twelve-lead electrocardiogram signal sequences. The electrocardiogram obtaining unit 300 may be an electrocardiograph machine. Preferably, the electrocardiogram obtaining unit 300 may be a twelve-lead electrocardiograph machine, which includes ten electrode patches. More than two electrode patches are placed on the limbs and measured in pairs. The twelve sets of lead potential changes on the body surface are recorded, and twelve sets of lead signals are drawn onto the electrocardiogram paper to obtain the twelve-lead electrocardiogram signal sequences.

The non-transitory machine-readable medium 400 is connected to the electrocardiogram obtaining unit 300 by at least one signal, and the non-transitory machine-readable medium 400 is configured for storing a program. The program is executed by at least one processing unit to obtain a prediction result. The prediction result is the stroke rate of the subject. The program includes a reference database obtaining module 410, a reference feature selecting module 420, a training module 430, a target feature selecting module 440 and a comparing module 450.

The reference database obtaining module 410 is configured for obtaining the reference database. The reference database includes a plurality of reference twelve-lead electrocardiogram signal sequences. Furthermore, the reference twelve-lead electrocardiogram signal sequences can be preliminary classified into an abnormal data and a non-abnormal data and labeled, so as to divide the reference database into two categories.

The reference feature selecting module 420 is configured for selecting at least one reference feature value according to the reference database. The reference feature value includes an image interval where an electrocardiogram signal curvature changes the most obtained by calculating a peak-to-peak time difference in the reference twelve-lead electrocardiogram signal sequences with a calculating unit 421.

The training module 430 includes a long short term memory 432. The long short term memory 432 is configured for storing an electrocardiogram signal real-time value and calculating a correlation between the feature value and the electrocardiogram signal real-time value. The long short term memory 432 is updated when the correlation exceeds the first preset threshold, and the atrial fibrillation prediction model is obtained when training reaches convergence. The long short term memory 432 can further include the forget gate, the input gate and the output gate. The forget gate is to filter the electrocardiogram signal real-time value whose curvature changes excessively to obtain the input value. The input gate is to input the input value, and the correlation is calculated by the Sigmoid function. The output gate is to calculate the correlation by the Sigmoid function to obtain the output value, and the output value is added to the long short term memory 432 when the output value exceeds the second preset threshold. Preferably, the forget gate, the input gate and the output gate can be concatenated bi-directionally, and the first preset threshold and the second preset threshold can be determined by the tan h function. Furthermore, the long short term memory 432 can be the bi-directional long short term memory.

The target feature selecting module 440 is configured for analyzing the target twelve-lead electrocardiogram signal sequence to obtain a target feature value. The target feature value includes an image interval where a target electrocardiogram signal curvature changes the most obtained by calculating a peak-to-peak time difference in the target twelve-lead electrocardiogram signal sequence with another calculating unit 441.

The comparing module 450 is configured for analyzing and comparing the target feature value and the reference feature value with the atrial fibrillation prediction model, so as to obtain a preset result. The preset result is the stroke rate of the subject in 3-6 months, which is a reference for physicians to diagnose, and the rate is 0% to 100%.

### Example

I. Reference Database

The reference database used in the present disclosure includes the clinical contents of the subjects after pseudonymization from 2009/01/01 to 2018/12/31, which is collected by China Medical University & Hospital with a retrospective manner. It is a clinical trial project approved by China Medical University & Hospital Research Ethics Committee with the number: CMUH107-REC2-134 (AR-1). The data is from a searching method of keyword parameters in the GE Healthcare MUSE system. The electrocardiography (ECG/EKG) waveform data of the patients with atrial fibrillation, myocardial infarction, etc. is collected, including twelve-lead electrocardiogram signal sequences. The original data is in Extensible Markup Language (XML) format. There is no particular restriction on the gender of the subjects whose images are collected, and there is no particular age range thereof. The reference subjects include 5,000 reference subjects without atrial fibrillation and 10,012 reference subjects with atrial fibrillation, with a total of 15,012 reference subjects. The abovementioned numbers are the “numbers of data” that actually used. The possibility of “examinations on the same patient” at “different timepoints/dates” is not ruled out.

II. Determining Stroke Rate of Subject

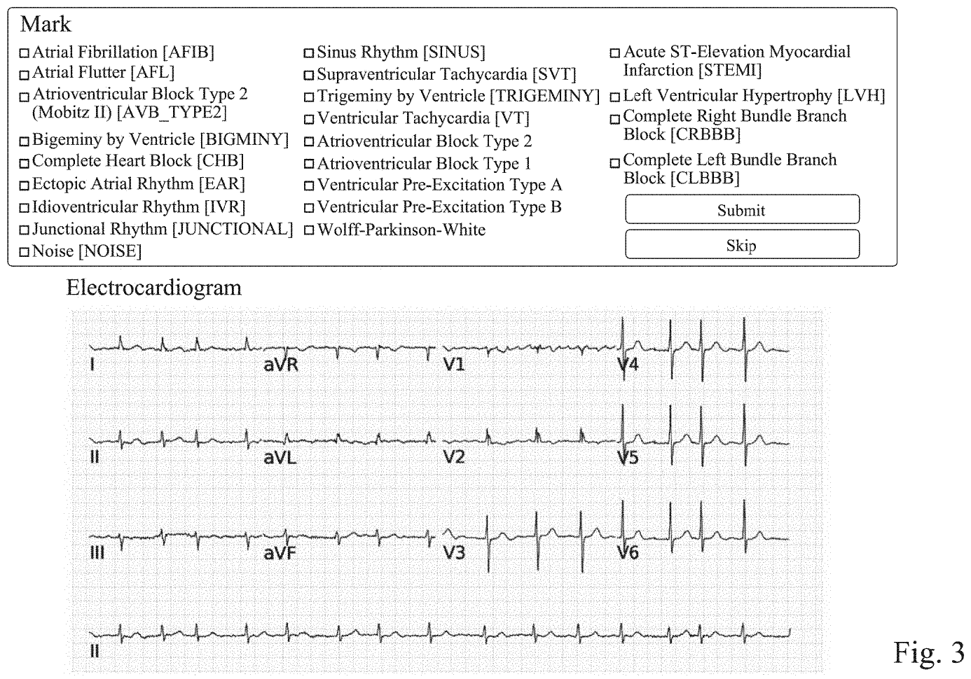

In this example, the optimized atrial fibrillation prediction model is established. First, the reference database is obtained. The reference database includes a plurality of reference twelve-lead electrocardiogram signal sequences. The reference twelve-lead electrocardiogram signal sequences are preliminary classified into an abnormal data and a non-abnormal data and labeled. Please refer to FIG. 3, which is a schematic diagram of data labeling platform of a reference database of the atrial fibrillation prediction model of the present disclosure. In order to make the atrial fibrillation prediction model, which will be established later, correctly learn the diseases corresponding to the twelve-lead electrocardiogram signal sequences, a data labeling platform is established without providing any personal information related to the patients and with restriction on specific connections. Physicians will use this platform to mark the reference database with multiple labels as a learning reference for the atrial fibrillation prediction model.

Then, the reference feature selecting module is used to select at least one feature value according to the reference database. The feature value includes an image interval where an electrocardiogram signal curvature changes the most obtained by calculating a peak-to-peak time difference in the reference twelve-lead electrocardiogram signal sequences with the calculating unit.

Then, the training step is performed. The bi-directional long short term memory network structure is used for neural network learning, and the machine will learn time series signals from the neural directions with different items. When optimizing parameters, the traditional recurrent neural network (RNN) uses the gradient descent method to optimize the parameter updating method. The method of finding the parameter change thereof is the backward propagation algorithm, but this algorithm will cause gradient explosion and gradient vanish due to the taken parameters. The forget gate is added into the atrial fibrillation prediction model of the present disclosure during training. Therefore, if a gradient explosion happens in the backward propagation algorithm, it can be blocked by the forget gate. When the input value is close to zero (that is, the value after the tenth decimal place) after calculated with mathematical formula, it will be directly ignored by the computer and causes gradient vanish. The pass gate can be used to pass the message on to avoid the gradient vanish.

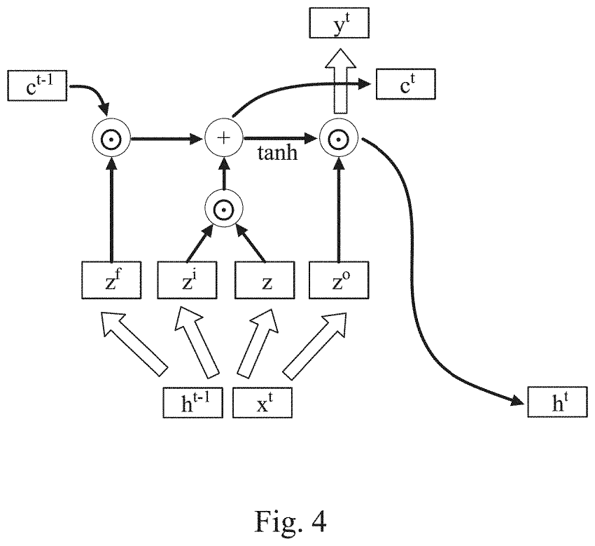

In detail, in the training step, the long short term memory is used to store the electrocardiogram signal real-time value and the correlation between the feature value and the electrocardiogram signal real-time value is calculated. The long short term memory is updated when the correlation exceeds the first preset threshold. Please refer to FIG. 4, which is a schematic diagram of a structure of a long short term memory of the atrial fibrillation prediction model of the present disclosure. The long short term memory uses a memory branch which is updated over time to improve the current decision result. The long short term memory includes the forget gate, the input gate, and the output gate for determining whether the memory should be updated or not. The forget gate, the input gate and the output gate is concatenated bi-directionally. The forget gate is to filter the electrocardiogram signal real-time value whose curvature changes excessively to obtain the input value. In detail, the forget gate uses the calculated zf (f stands for forget) for the forget gating, so as to control which ct-1 in the previous state should be left or forgotten, usually a Sigmoid function. The input gate is to input the input value, and the correlation is calculated by the Sigmoid function. In detail, the input gate determines whether the current input and the new generated memory cell candidate should be added into the long term memory. The input gate also uses the Sigmoid function to indicate whether they are added or not. Specifically, the input xt is selected to be memorized. Which is important will be fully recorded, and which is not important will be partially recorded. The current input content is represented by the previously calculated z. The selected gating signal is controlled by zi (i stands for information). The output gate is to calculate the correlation by the Sigmoid function to obtain the output value, and the output value is added to the long short term memory when the output value exceeds the second preset threshold. In detail, the output gate determines which will be the output of the current state. It is mainly controlled by zo. The co obtained in the previous stage is changed through a tan h activating function. Please refer to formula (I), formula (II) and formula (III) for detailed calculation of the forget gate, the input gate and the output gate.

c^{t}=z^{f}⊙c^{t-1}+zⁱ⊙z Formula (I);

h^{t}=z^{o}⊙ tan h(c^{t}) Formula (II);

y^{t}=σ(W′h^{t}) Formula (III).

Wherein, the first preset threshold and the second preset threshold are determined by the tan h function. The output value of the tan h function is between −1 and 1, which is the preset value from calculating a large number of twelve-lead electrocardiogram signal sequences with mathematical formula of machine learning. The atrial fibrillation prediction model is obtained when training reaches convergence and the preset result is obtained, and the preset result is the stroke rate of the subject.

During the training of the atrial fibrillation prediction model, when the correlation between the feature value and the electrocardiogram signal real-time value exceeds the first preset threshold, the long short term memory is updated to reach convergence and obtain the atrial fibrillation prediction model. When the correlation is closer to −1, the probability that the subject does not have atrial fibrillation is higher. On the contrary, when the correlation is close to 1, the probability that the subject has atrial fibrillation is higher. When predicting atrial fibrillation with the atrial fibrillation prediction model, the forget gate will first filter the electrocardiogram signal real-time value to obtain the input value, and the correlation calculated by the Sigmoid function is input through the input gate. The output gate will calculate the correlation by the Sigmoid function to obtain the output value. When the output value exceeds the second preset threshold, the output value is added to the long short term memory. When the output value is closer to −1, the probability that the subject does not have atrial fibrillation is higher. On the contrary, when it is close to 1, the probability that the subject has atrial fibrillation is higher.

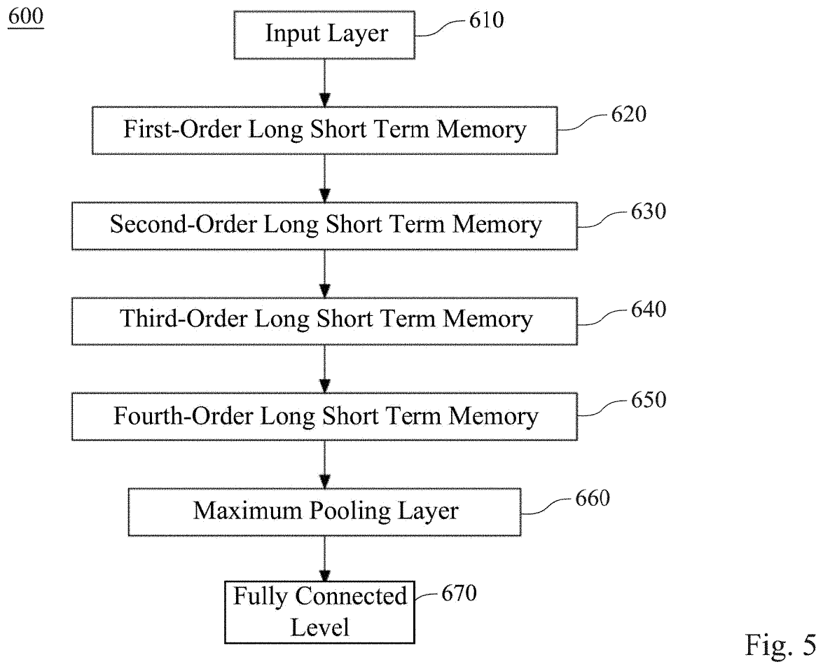

Furthermore, please refer to FIG. 5, which is a structural diagram of the long short term memory 600 of the atrial fibrillation prediction model of the present disclosure. The long short term memory 600 of the atrial fibrillation prediction model of the present disclosure is a four-order long short term memory group with 128*4 long short term memory inside, which includes an input layer 610, a first-order long short term memory 620, a second-order long short term memory 630, a third-order long short term memory 640, a fourth-order long short term memory 650, a maximum pooling layer 660 and a fully connected level 670. Each of the first-order long short term memory 620, the second-order long short term memory 630, the third-order long short term memory 640 and the fourth-order long short term memory 650 has 128 long short term memories. The first-order long short term memory 620 can process the feature values with low complexity, the second-order long short term memory 630 can process the feature values with slight complexity, the third-order long short term memory 640 can process the feature values with higher complexity, and the fourth-order long short term memory 650 can process the feature values with highest complexity. The maximum pooling layer process the collection according to the features of four-order long short term memory learning. The fully connected layer (Sigmod function/tan h function) will output the final results according to the feature learning.

In this example, the atrial fibrillation prediction system with the established atrial fibrillation prediction model is further used to predict stroke of the subject. The steps are as follows. The established atrial fibrillation prediction model is provided. The target twelve-lead electrocardiogram signal sequence of the subject is provided. The target feature value is obtained by analyzing the target twelve-lead electrocardiogram signal sequence with the target feature selecting module. Finally, the comparing module is used to analyze and compare the target feature value and the reference feature value with the atrial fibrillation prediction model, so as to obtain the preset result and predict the stroke rate of the subject.

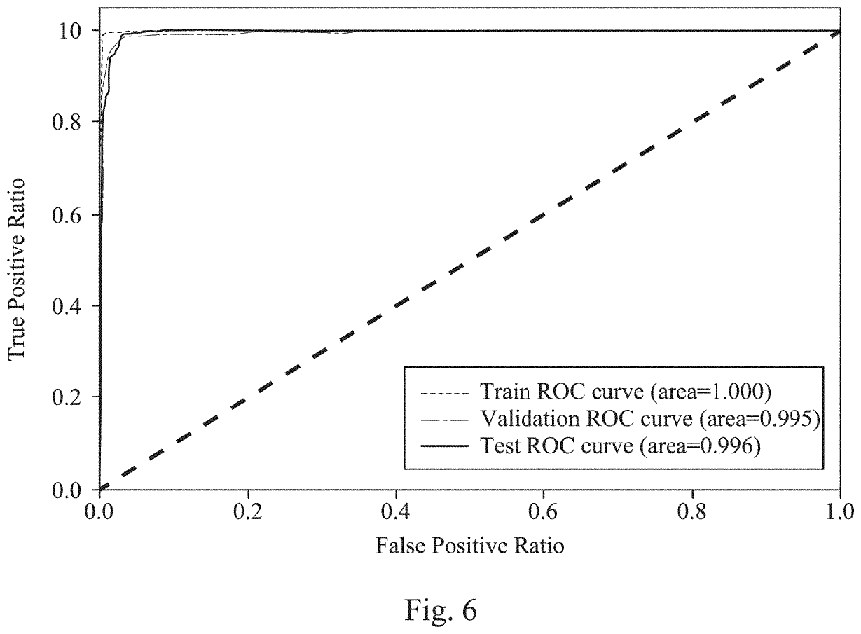

Please refer to FIG. 6, which is a diagram of receiver operating characteristic curve (ROC) for predicting a subject's stroke rate of the atrial fibrillation prediction system of the present disclosure. The results show that as predicting the stroke rate of the subject with the atrial fibrillation prediction model of the present disclosure, the area under the curve (AUC) of the test is 0.996 and the ROC value is 99.6%. It is shown that the atrial fibrillation prediction model and the atrial fibrillation prediction system of the present disclosure can accurately predict the stroke rate of the subject with the twelve-lead electrocardiogram signal sequences.

In this regard, an atrial fibrillation prediction model and an atrial fibrillation prediction system are provided in the present disclosure. The long short term memory network structure is used for neural network learning, and the machine will learn time series signals from the neural directions with different items, which can objectively and accurately determine whether the subject has atrial fibrillation with the twelve-lead electrocardiogram signal sequences and the stroke rate thereof can be further predicted. A second opinion can be provided to specialists, so as for helping physicians to judge in clinical practice. It only takes 0.1-1 seconds on average from inputting the original image to determining the result, and the accuracy is as high as 0.996. Thus, based on the atrial fibrillation prediction model and the atrial fibrillation prediction system of the present invention, the twelve-lead electrocardiogram signal sequences of a case can be automatically and rapidly analyzed, which helps the health professional to judge and diagnose at an early stage, and enhance the discovery rate of early stroke, in order for physicians to plan the follow-up treatment for the patients.

Although the present disclosure has been described in considerable detail with reference to certain embodiments thereof, other embodiments are possible. Therefore, the spirit and scope of the appended claims should not be limited to the description of the embodiments contained herein. It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

1. An atrial fibrillation prediction model, comprising the following establishing steps:
obtaining a reference database, wherein the reference database comprises a plurality of reference twelve-lead electrocardiogram signal sequences;
performing a feature selecting step to select at least one feature value according to the reference database, wherein the at least one feature value comprises an image interval where an electrocardiogram signal curvature changes the most obtained by calculating a peak-to-peak time difference in the reference twelve-lead electrocardiogram signal sequences with a calculating unit; and
performing a training step to store an electrocardiogram signal real-time value by a long short term memory and calculate a correlation between the at least one feature value and the electrocardiogram signal real-time value, wherein the long short term memory is updated when the correlation exceeds a first preset threshold, and the atrial fibrillation prediction model is obtained when training reaches convergence and a preset result is obtained.

2. The atrial fibrillation prediction model of claim 1, wherein the long short term memory is a bi-directional long short term memory.

3. The atrial fibrillation prediction model of claim 1, wherein the long short term memory further comprises:
a forget gate to filter the electrocardiogram signal real-time value whose curvature changes excessively to obtain an input value;
an input gate to input the input value, wherein the correlation is calculated by a Sigmoid function; and
an output gate to calculate the correlation by the Sigmoid function to obtain an output value, wherein the output value is added to the long short term memory when the output value exceeds a second preset threshold.

4. The atrial fibrillation prediction model of claim 3, wherein the forget gate, the input gate and the output gate are concatenated bi-directionally.

5. The atrial fibrillation prediction model of claim 3, wherein the first preset threshold and the second preset threshold are determined by a tan h function.

6. An atrial fibrillation prediction system, comprising:
an electrocardiogram obtaining unit configured for obtaining a target twelve-lead electrocardiogram signal sequence; and
a non-transitory machine-readable medium connected to the electrocardiogram obtaining unit by at least one signal, wherein the non-transitory machine-readable medium is configured for storing a program, the program is executed by a processing unit to obtain a prediction result, and the program comprises:
a reference database obtaining module configured for obtaining a reference database, wherein the reference database comprises a plurality of reference twelve-lead electrocardiogram signal sequences;
a reference feature selecting module configured for selecting at least one reference feature value according to the reference database, wherein the at least one reference feature value comprises an image interval where an electrocardiogram signal curvature changes the most obtained by calculating a peak-to-peak time difference in the plurality of reference twelve-lead electrocardiogram signal sequences with a calculating unit;
a training module, comprising:
a long short term memory configured for storing an electrocardiogram signal real-time value and calculating a correlation between the at least one reference feature value and the electrocardiogram signal real-time value, wherein the long short term memory is updated when the correlation exceeds a first preset threshold, and an atrial fibrillation prediction model is obtained when training reaches convergence;
a target feature selecting module configured for analyzing the target twelve-lead electrocardiogram signal sequence to obtain a target feature value, wherein the target feature value comprises an image interval where a target electrocardiogram signal curvature changes the most obtained by calculating a peak-to-peak time difference in the target twelve-lead electrocardiogram signal sequence with another calculating unit; and
a comparing module configured for analyzing and comparing the target feature value and the at least one reference feature value with the atrial fibrillation prediction model, so as to obtain a preset result.

7. The atrial fibrillation prediction system of claim 6, wherein the long short term memory is a bi-directional long short term memory.

8. The atrial fibrillation prediction system of claim 6, wherein the long short term memory further comprises:
a forget gate configured for filtering the electrocardiogram signal real-time value whose curvature changes excessively to obtain an input value;
an input gate configured for inputting the input value, wherein the correlation is calculated by a Sigmoid function; and
an output gate configured for calculating the correlation by the Sigmoid function to obtain an output value, wherein the output value is added to the long short term memory when the output value exceeds a second preset threshold.

9. The atrial fibrillation prediction system of claim 8, wherein the forget gate, the input gate and the output gate are concatenated bi-directionally.

10. The atrial fibrillation prediction system of claim 8, wherein the first preset threshold and the second preset threshold are determined by a tan h function.
